(19) United States
(12) Patent Application Publication
(10) Pub. No.: US 2016/0249815 A1
(43) Pub. Date: Sep. 1, 2016

(54) SAFETY SYSTEM

(71) Applicant: Safe Family Wearables, LLC., Los Angeles, CA (US)

(72) Inventors: Merle Freeman, Marina Del Rey, CA (US); Jennifer Lazarus, Los Angeles, CA (US); Evan Lazarus, Los Angeles, CA (US)

(73) Assignee: Safe Family Wearables, LLC., Los Angeles, CA (US)

(21) Appl. No.: 15/055,459

(22) Filed: Feb. 26, 2016

Related U.S. Application Data
(60) Provisional application No. 62/121,467, filed on Feb. 26, 2015.

(51) Int. Cl. A61B 5/0205; H04B 17/318; H04W 4/02

(52) U.S. Cl. CPC A61B 5/0205; H04W 4/028; H04B 17/318; A61B 5/02438

(57) ABSTRACT

A safety system includes a wearable device having means to determine whether a child has been left in a car seat and further having means to detect the wearable device's global position and the ambient temperature, heart rate, and activity of a person wearing the wearable device. The wearable device can send notice to a smartphone or other designated device when certain specified conditions have been met or exceeded. The information monitored and/or gathered can be stored and retrieved as needed.

### BACKGROUND

1. Field of the Invention

The invention disclosed herein is wearable device that is part of a safety system for remotely monitoring the activity, condition, status, and location of a person as well as the status of their surroundings. The wearable device has means to communicate with a central server in the cloud. The wearable device is controlled by a smartphone application that communicates with the mobile device via the central server in the cloud. The wearable device includes sensors that gather information and send it to the central server at specified intervals and at any time an alarm condition has been met.

2. Description of the Related Art

The related art may include the following:

a. United States patent application Publication, Pub. No. US 2014/0118149 A1 discloses a mobile device for keeping track of a child at all times. Tracing application software is used to set boundaries using a communication device. A data server receives the transmitter signal from the mobile device and sends alert signals to the communication device. The tracing app can also directly contact local officials. The enclosure for the transmitter can be injection molded. The transmitter includes a GPS receiver.

b. United States patent application Publication, Pub. No. US 2010/0238033 A1 discloses a wearable tracking device that uses GPS, GSM or GPRS. GSM and GPRS enable the device to be tracked when the GPS is blocked from communicating with a satellite. If the device is removed, destroyed or tampered with, alerts may be sent. The device can be set to send an alarm when a specified boundary or speed limit is exceeded.

c. U.S. Pat. No. 8,525,670 B1 discloses a tracking system that includes a wearable, ring-shaped tracking device that has a radio frequency identification (RFID) circuit with a unique signal that automatically transmits its location to a first external transceiver. If the RFID signal is undetected, an alarm is sent. A GPS receiver and secondary transmitter are included as a back-up. The secondary transmitter can be Bluetooth or a transmitter using mobile phone data. The device may also include a Cardio Respiratory sensor which is in contact with the wearer's skin. When certain heartbeat parameters are met, an alarm can be sent. The device can also exclude RFID and embody only GPS.

d. U.S. Pat. No. 8,031,075 B2 discloses a wearable device that monitors the wearer's condition and the wearer's ambient conditions. Conditions that may be monitored include temperature, movement, force, sound, and ultra-violet rays.

Unlike the present invention, however, none of the prior art discloses a wearable device that, by itself, can determine if a child has been left in a car seat. Additionally, no single prior art reference includes the combination of features that are included in the present invention.

### SUMMARY OF THE INVENTION

The safety system of the present invention includes a wearable device having means to transmit information regarding the conditions or status of the person wearing the device and their surroundings. The wearable device includes software and hardware working in conjunction with sensors that detect and gather information. This information is then uploaded to a central server in the cloud. When the information detected or gathered by a sensor meets preset specified parameters, notice of the alarm condition is immediately and automatically sent to the central server which then sends the alarm notification to a smartphone or other designated device.

The information that can be gathered by sensors includes: (a) the global position of the device, (b) the proximity of the wearable device to a designated smartphone or to a specified or tagged location, (c) the heart rate of the wearer, (d) the ambient temperature around the person wearing the device, (e) the motion or activity of the wearer, (f) whether the distance between the wearer and a second device, such as a smartphone, is increasing, decreasing, or remaining constant, (g) whether the wearable device has been unclasped from the wearer, (h) the unique identification of the wearable device, (i) the battery level of the wearable device, and (j) the medical history and personal information of the wearer.

The wearable device can also include means for communicating via bluetooth. The wearable device may also include a Subscriber Identity Module (SIM) card to allow the wearable device to communicate via a cell phone network.

The wearable device may be charged via a micro-USB charging unit. The wearable device may be encased in a baby-safe medical grade silicon cover that keeps it waterproof and choke proof. The device may be attached to a child via a band with a childproof buckle or clip, keeping it securely on the child.

The centralized server in the cloud acts as a conduit between the wearable device and a mobile application (“app”). The central server can handle any number of wearable devices and smartphones, tablets, computers and/or other electronic devices that communicate with each wearable device. Each smartphone app can control any number of designated wearable devices. The typical scenario where this ability would be useful is for parents who have two or more children, each one of which has a wearable device.

Each wearable device may send data at specified intervals, e.g., every 30 seconds. Alternatively, the interval can be set so that the data is continuously sent in real time. All of the data can be stored for historical analysis and retrieval by the mobile app. Each device can be registered with a unique identification and security credentials for privacy.

The mobile application serves to provide the user with all of the information provided by the wearable device. This information is provided in a user friendly graphical user interface. The app allows the user to:

a. Select which wearable device(s) to monitor (using a unique identifier assigned to each wearable device),

b. Display the ambient temperature of the person wearing the wearable device, typically a child,

c. Display a map with the exact GPS location of the wearable device,

d. Set a geo-fence/perimeter or a boundary defined by a radius of a certain distance from a specified location,

e. Identify whether the child wearing the device is sitting, lying down or upright,

f. Identify whether the child wearing the device is in motion and the speed at which they are traveling,

g. Identify the child's heart rate,

h. Identify whether a child has been left in a car seat by determining whether (1) the bluetooth signal between the device and a designated smartphone is getting weaker, (2) the child's GPS location is changing over time, (3) the ambient temperature is increasing, and (4) the activity of the wearer is below a specified threshold,

i. Identify whether wearable device's battery needs recharging,

j. Register the wearable device and assign users, and

k. Make in-app purchases.

The wearable device also uses bluetooth signal strength to determine whether the distance between the wearable device and a designated smartphone has increased beyond a specified distance. The app can then notify the smartphone operator that the distance between the smartphone and the wearable device has exceeded the specified distance. For example, if this parameter is set to 30 feet and the distance between the smartphone and the wearable device exceeds 30 feet, notice in this regard in the form of a phone call, text message, email or alert will be sent to the smartphone.

### BRIEF DESCRIPTION OF THE DRAWINGS

FIG. 1 is a perspective view of a wearable device.

FIG. 2 is a perspective exploded view of a wearable device.

FIG. 3 is a perspective exploded view of a wearable device.

FIG. 4 is a perspective exploded view of a wearable device.

FIG. 5 is a perspective magnified exploded view of a wearable device.

FIG. 6 is a perspective view of the electronics of a wearable device.

FIG. 7 is a perspective exploded view of the electronics, base housing, housing cover, buckle, and printable band of a wearable device.

FIG. 8 is a perspective view of a wearable device showing the buckle and overlay clip.

FIG. 9(a) is a perspective view of the outer side of a buckle and overlay clip of a wearable device.

FIG. 9(b) is a perspective view of the inner side of a buckle and overlay clip of a wearable device.

FIG. 10 is a perspective view of the housing cover of a wearable device.

FIG. 11 is a perspective view of the inner side of a buckle and overlay clip of a wearable device.

FIG. 12 is a perspective view of the inner side of a buckle and overlay clip of a wearable device.

FIG. 13 is a perspective view of the inner side of a buckle and overlay clip of a wearable device.

FIG. 14 is a perspective view of the inner side of a buckle and overlay clip of a wearable device.

FIG. 15 is a perspective view of the outer side of a buckle and overlay clip of a wearable device.

FIG. 16 shows a sample home screen for an app for a wearable device.

FIG. 17 shows a sample launch screen for an app for a wearable device.

FIG. 18 shows a sample menu screen for an app for a wearable device.

FIG. 19 shows a sample summary screen for an app for a wearable device.

FIG. 20 shows a sample temperature screen for an app for a wearable device.

FIG. 21 shows a sample GPS screen for an app for a wearable device.

FIG. 22 shows a sample heart rate screen for an app for a wearable device.

FIG. 23 shows a sample activity screen for an app for a wearable device.

### DETAILED DESCRIPTION OF THE INVENTION

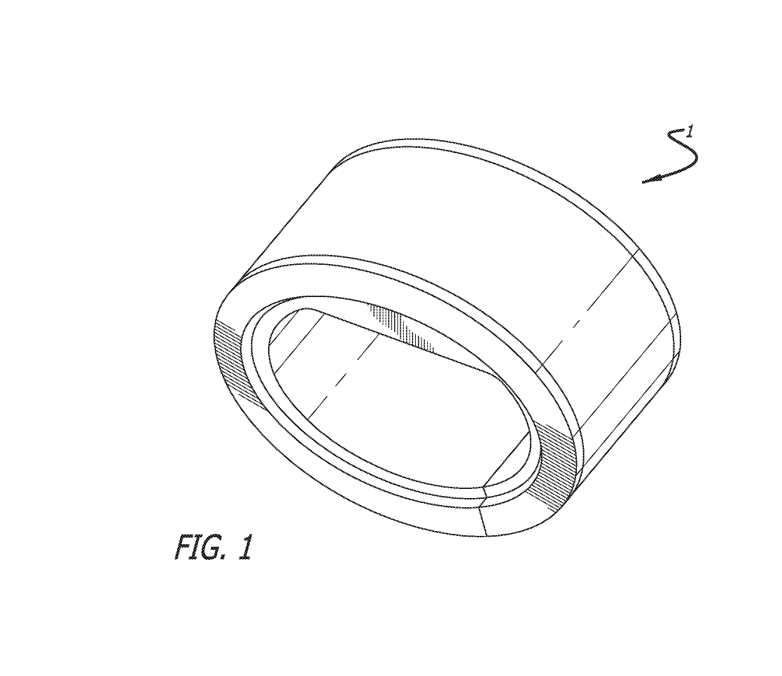

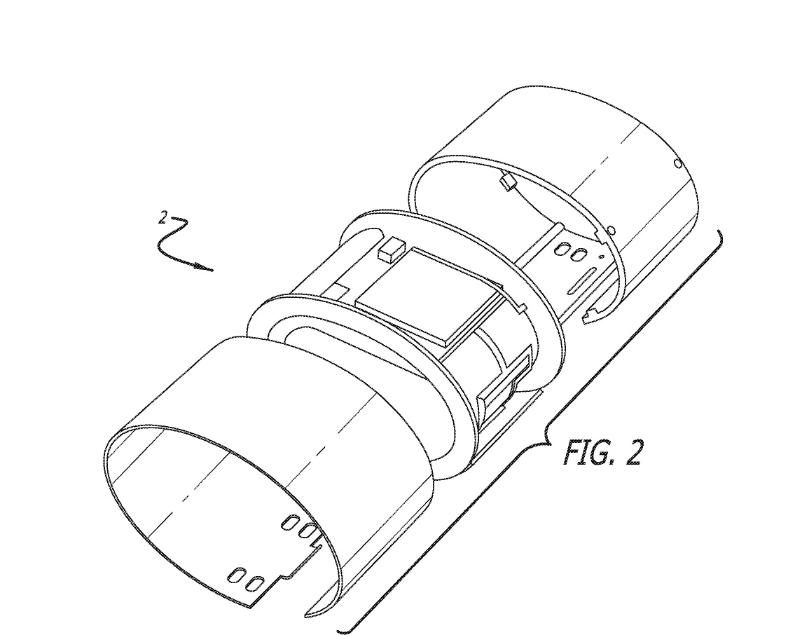

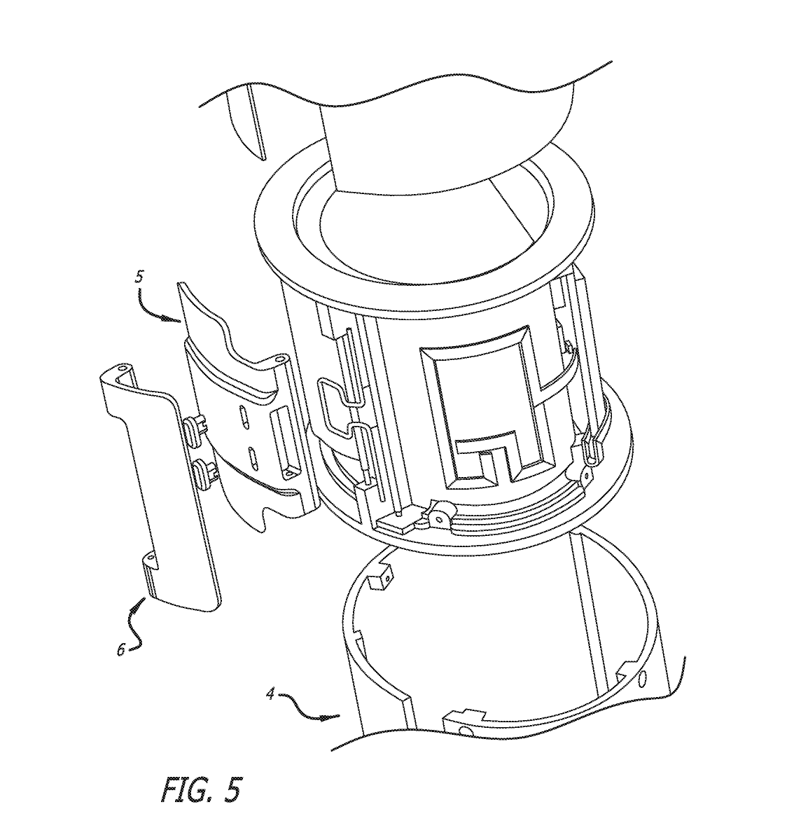

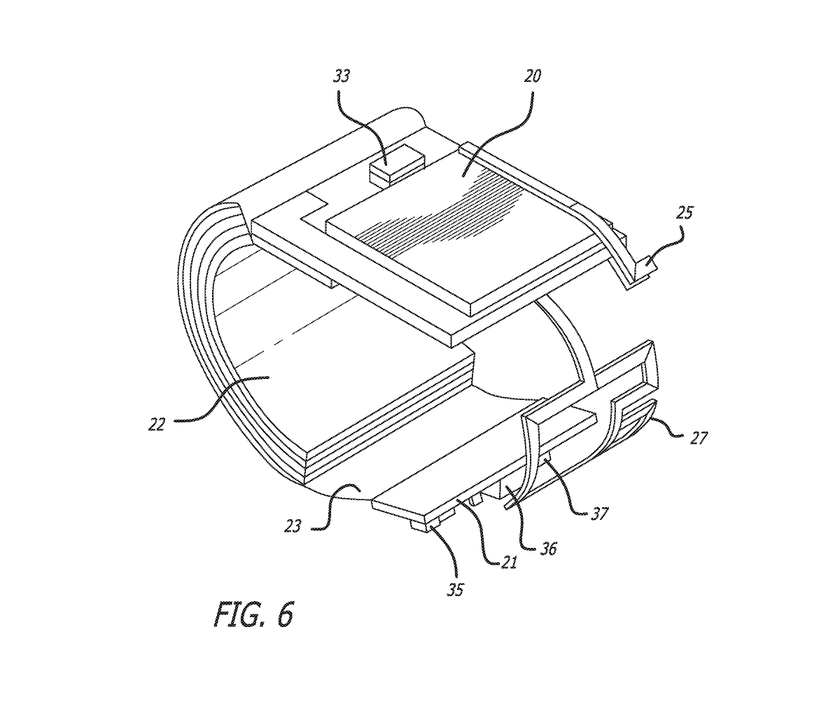

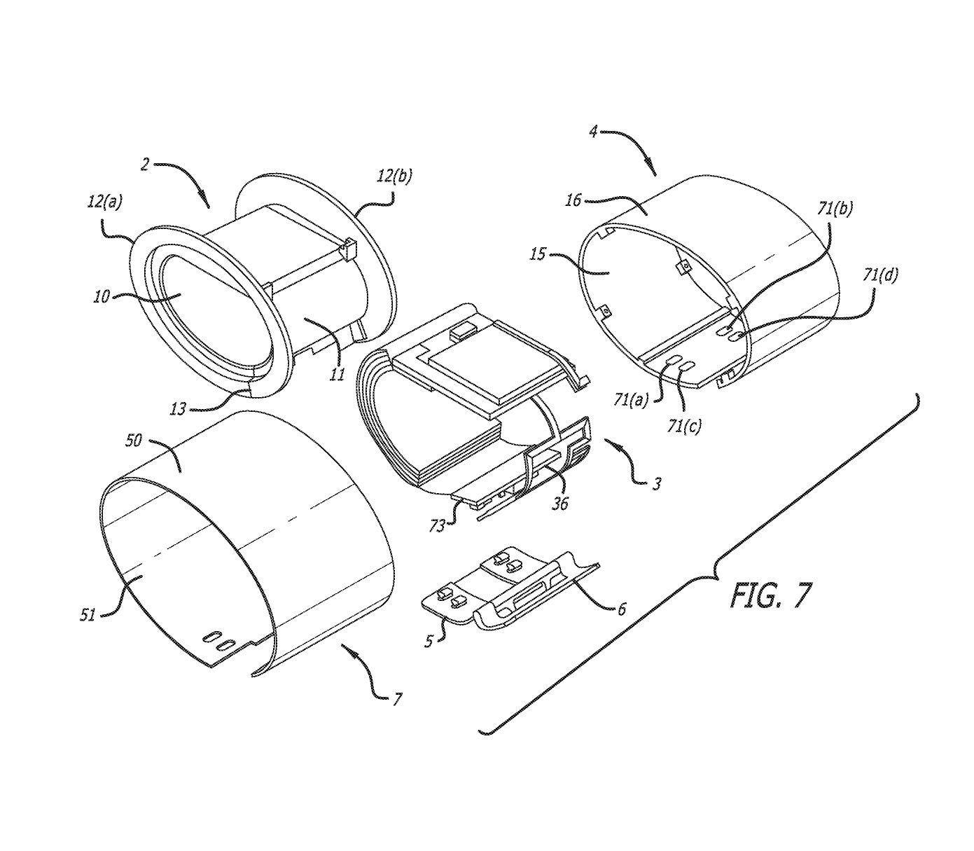

FIGS. 1-5 show a sample embodiment of a wearable device 1. FIGS. 6-7 show a sample configuration and layout for the components of a wearable device 1. FIGS. 8-15 show an example of a buckle 5 and overlay clip 6 of a wearable device 1.

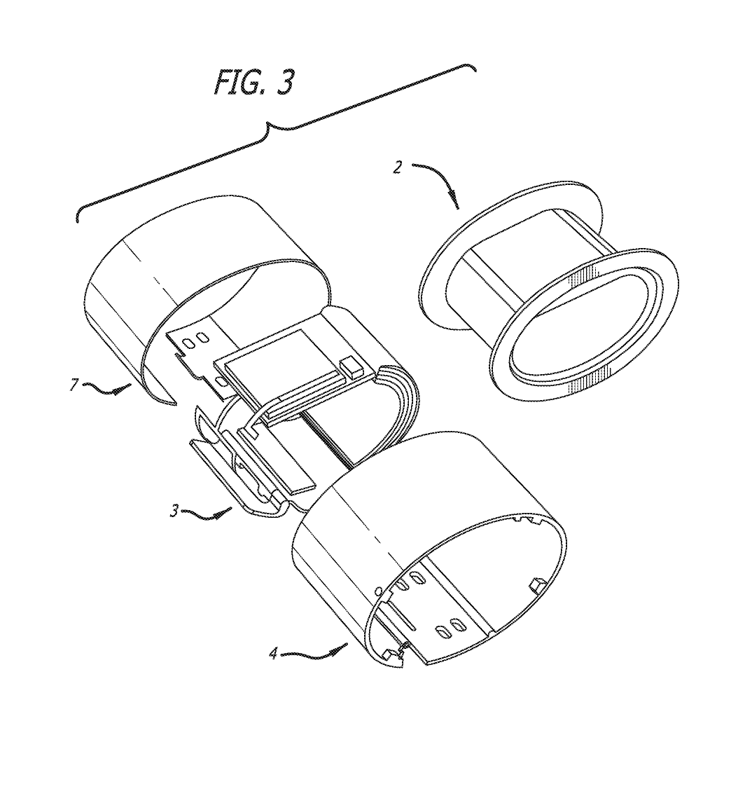

The wearable device 1 (FIG. 1) comprises a base housing 2 (FIG. 3) having an inner side 10, an outer side 11, retainer ridges 12(a), 12 (b), and an opening 13 (FIG. 7). An electronics layer 3 (FIG. 7) substantially surrounds the outer side 11 of the base housing 2. A housing cover 4 (FIG. 7) substantially surrounds the electronics layer 3. The housing cover 4 comprises an inner side 15 and an outer side 16. A buckle 5 (FIG. 7) combines with an overlay clip 6 to provide means to lock and unlock the opening 13. The buckle 5 is attachable to the housing cover 4. A printable band 7 (FIG. 7) substantially surrounds the housing cover 4. The printable band 7 comprises an outer side 50 and an inner side 51. In lieu of or in addition to the printable band 7, an anti-bacterial film (not shown) and bacterial detection film (not shown) may be affixed to the housing cover 4.

The electronics layer 3 comprises a primary board 20, secondary board 21, battery pack 22, and a flexboard 23 (FIG. 6) that electrically connects the primary board 20 to the secondary board 21.

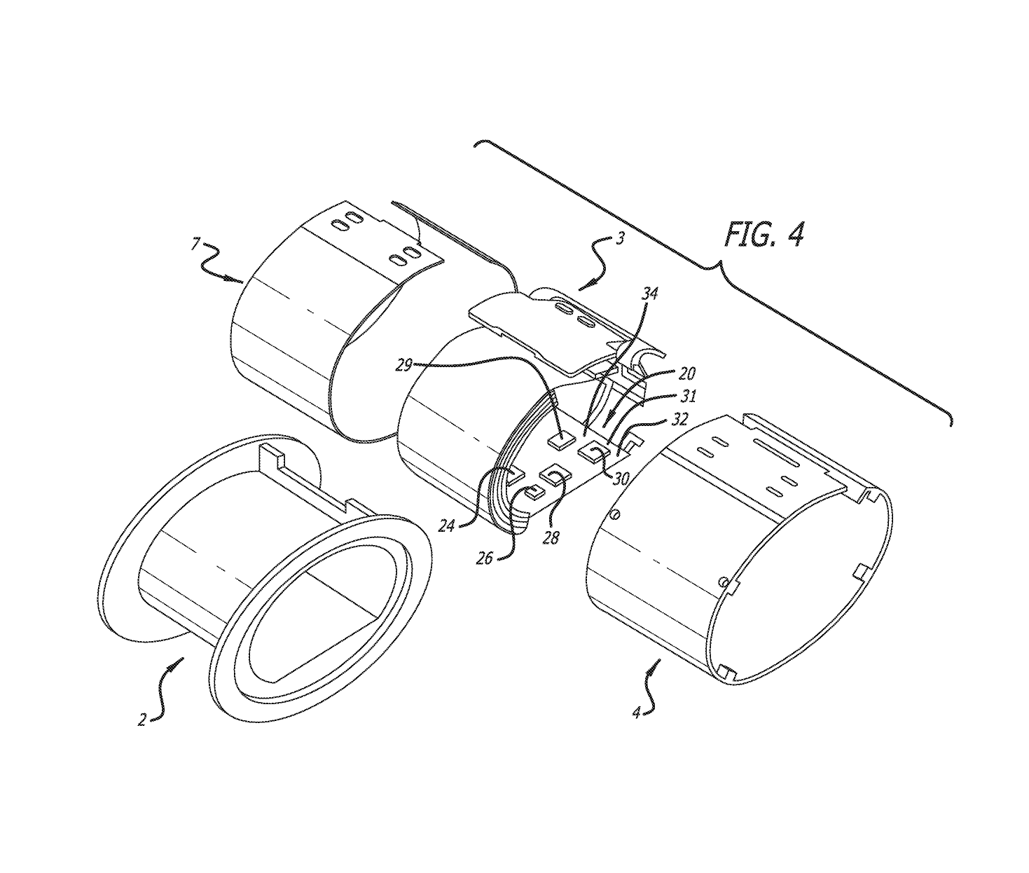

The primary board 20 comprises a Telit HE910 chip which includes a GPS (Global Positioning System) modem 24 (FIG. 4) and a GSM (Global System for Mobile Communications) chip 26 (FIG. 4). The primary board also comprises a GPS antenna 25 (FIG. 6), GSM antenna 27 (FIG. 6), a SIM card 28 (FIG. 4), an optical heart rate monitor 29 (FIG. 4), an accelerometer 30 (FIG. 4), an ambient temperature sensor 31 (FIG. 4), flash memory 32 (FIG. 4), and a bluetooth module 33 (FIG. 4). The primary board 20 further comprises a microcontroller 34 (FIG. 4) which controls all of the other components on the primary board 20.

The secondary board 21 comprises a power module 35 (FIG. 6) to monitor charging of the battery pack 22, a micro USB port 36 (FIG. 6) to charge the battery pack 22, and a battery gauge 37 (FIG. 6) to indicate the remaining battery power.

The buckle 5 comprises and on/off actuator 38 (FIG. 13) that sends a signal whenever the buckle 5 has been unbuckled or opened. When the buckle 5 is closed, the on/off actuator 38 depresses an actuator surface 73 (FIG. 7) to indicate that the wearable device 1 has been properly closed

Information gathered from the GPS modem 24, GSM chip 26, heart rate monitor 29, accelerometer 30, ambient temperature sensor 31, flash memory 32, power module 35, battery gauge 37, and on/off actuator 38 is all sent to the microcontroller 34. The SIM card 28 is preset to connect to a specified cell phone network. That connection is directed to a mobile virtual network operator (MVNO) which is dedicated for operation and control of the wearable device 1. The MVNO allows for cellular connectivity to allow data to be pushed to a cloud server. Once the SIM card 28 makes the cell phone connection, the microcontroller 34 sends the information to the MVNO. The mobile app on the smartphone or a tablet is used to control and interact with the wearable device 1 via the MVNO. The flash memory 32 holds the interval settings and alarm settings as specified by the mobile app operator.

The wearable device 1 is preset to send notice, in the form of a telephone call, text message, and/or email, to a designated smartphone or other device whenever a child wearing the wearable device 1 has been left in a car. Notice that a child has been left in a car is activated whenever (1) the bluetooth signal strength between the wearable device 1 and the smartphone or other device is getting weaker, (2) the ambient temperature, as determined by the temperature sensor 31 is increasing, (3) activity of the wearer, as determined by the accelerometer 30 falls below a preset threshold, and (4) the GPS location is unchanged. The wearable device 1 is preset to detect all four of these conditions simultaneously.

The mobile app allows a person to control the interval at which information from the wearable device 1 is sent to the central server. The operator of the mobile app can then access that information after each interval period has transpired. The information in this regard is also stored for archival purposes. For example, the operator of the mobile app could pull up a map to see all of the locations where the wearable device 1 has been during the past month. In helicopter mode, the interval period is replaced with real-time, continuous monitoring.

The mobile app allows the operator to set alarm conditions that, if met, will result in the wearable device 1 immediately sending a notice to the smartphone or other designated device that the condition has been met. For example, if the operator specifies an interval of 30 minutes and sets the heart rate monitor to send an alert or notice if the heart rate exceeds 100 beats per minute, the alarm setting will be acted upon and sent immediately regardless of any interval setting each time the heart rate exceeds 100 beats per minute.

The mobile app can also be used to tag a specific geographic location and set a radius around that tagged location such that if the wearable device 1 moves outside that boundary, an alarm or notice is sent to a designated smartphone or other designated device. A location might be tagged in this manner, for example, when a parent drops off a child at a particular location. That location can then be saved by the mobile app and a perimeter boundary specified. If the wearable device 1 moves outside that boundary, an alarm or notice is sent to the designated smartphone or other device. A location can also be tagged and a boundary set around it without the mobile app operator actually being present at the tagged location.

Using the mobile app, a graphical icon can be used as an overlay on a map to indicate a tagged location. In another location on the same screen, the mobile app can provide a user input area where the operator can specify a radius, in feet, extending around the tagged location. Once entered, that information is sent to the flash memory 32 of the wearable device 1 and thereby becomes a condition for an alarm or notice to be sent if the specified radius is exceeded.

The wearable device can detect ambient temperature surrounding the wearer and can be set so that if a specified ambient temperature is reached, the wearable device 1 will send an alert, phone call, email, text, and/or other form of notice to a designated cell phone, land line, computer, tablet, and/or other electronic device.

The mobile app can present ambient temperature on a dedicated temperature screen. In another location on the same screen, a temperature that, if met, would trigger an alarm notice, can be set by simply swiping a finger up or down over the indicated temperature setting to, respectively, increase it or decrease it.

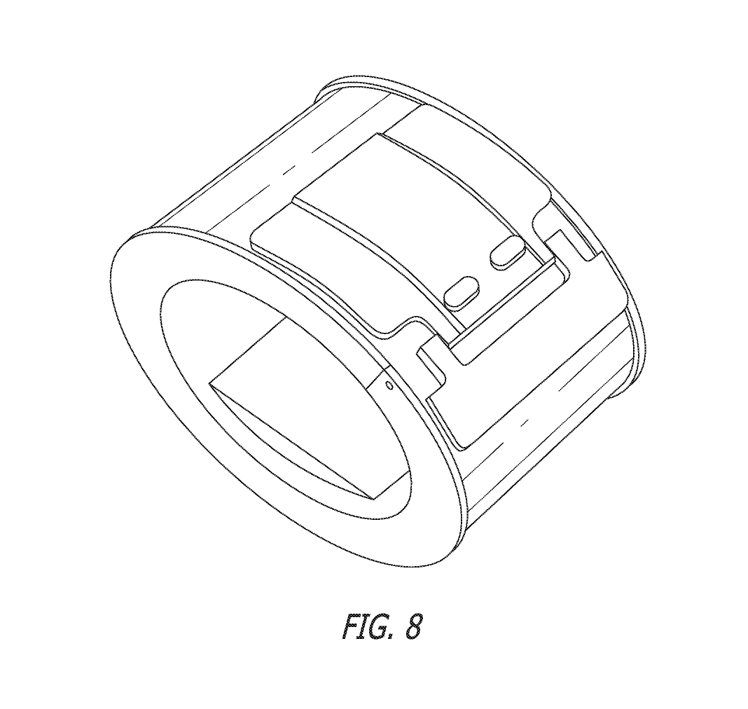

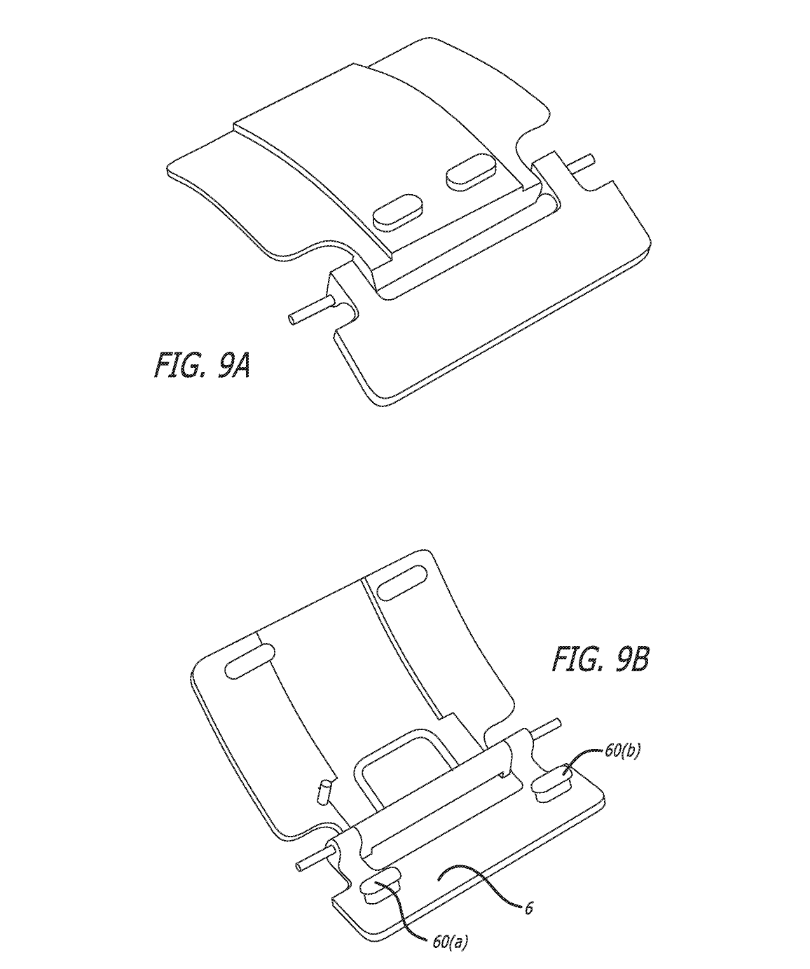

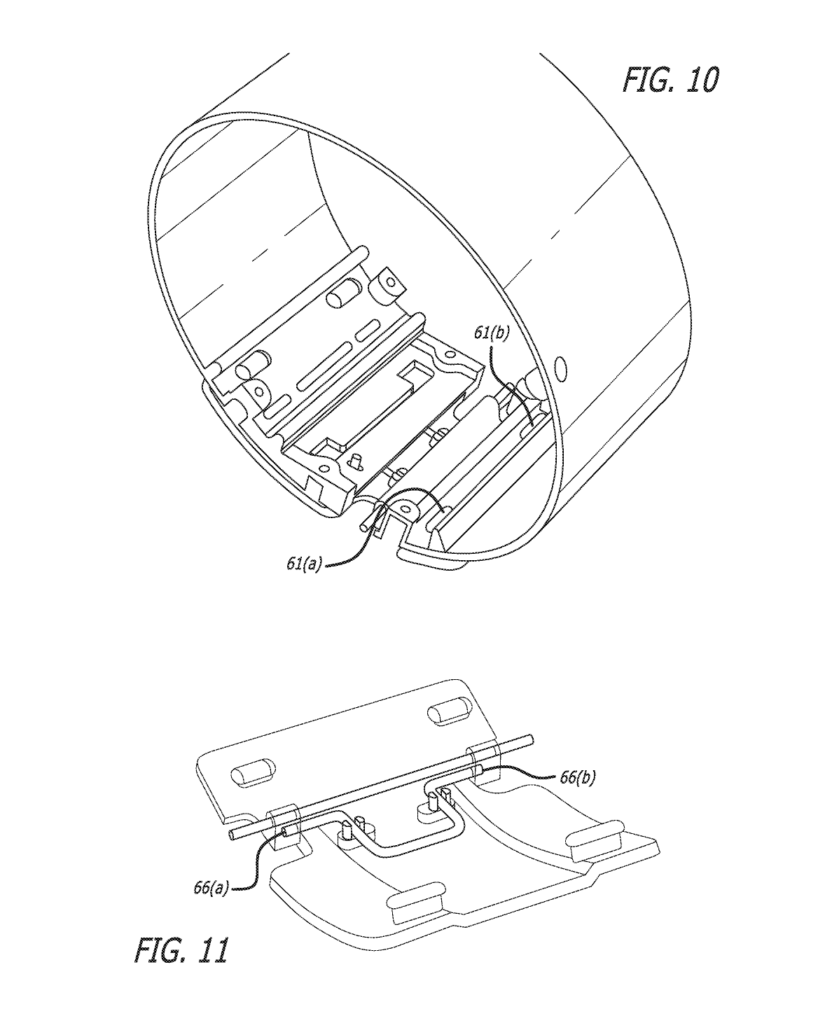

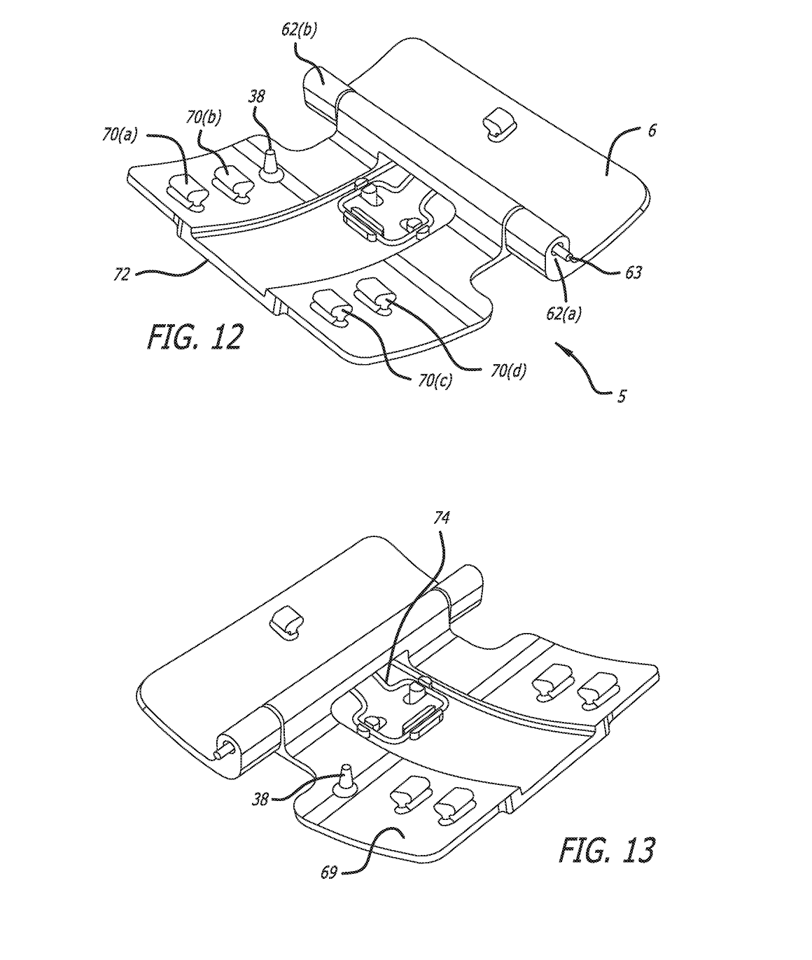

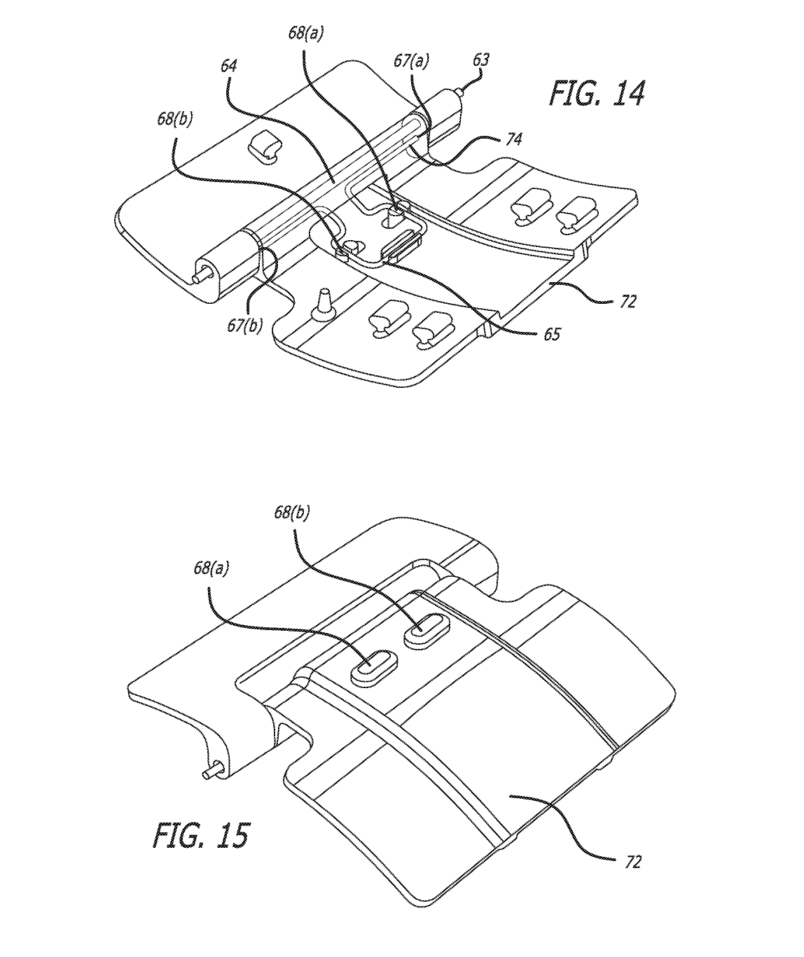

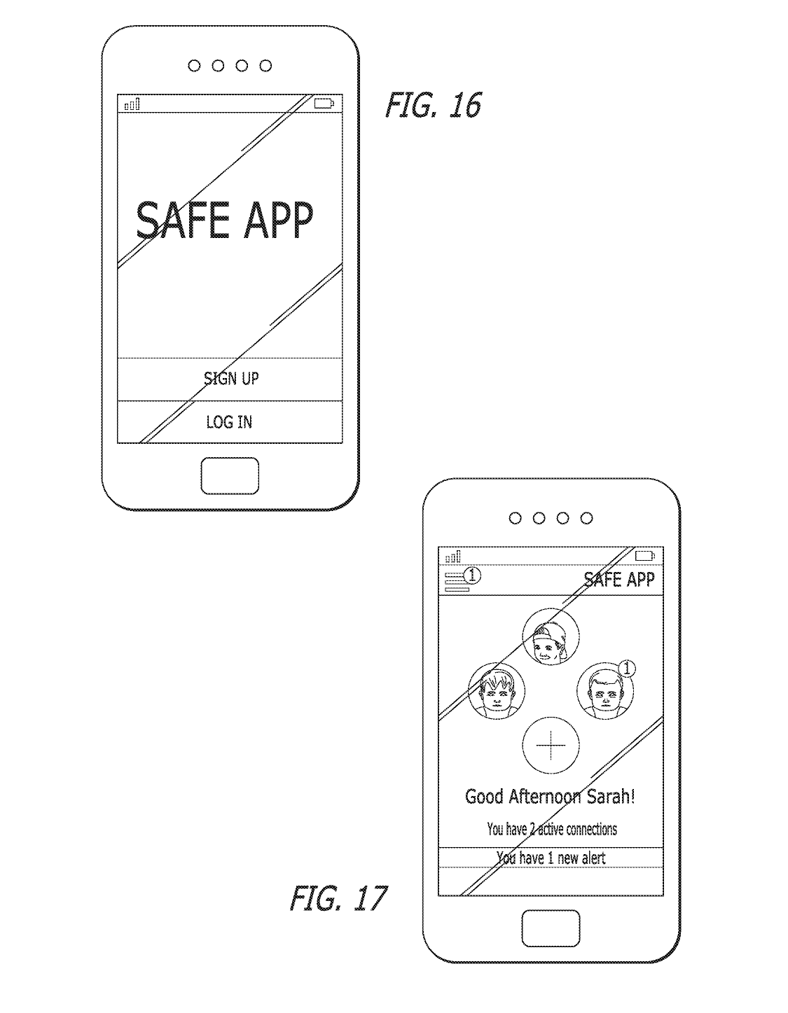

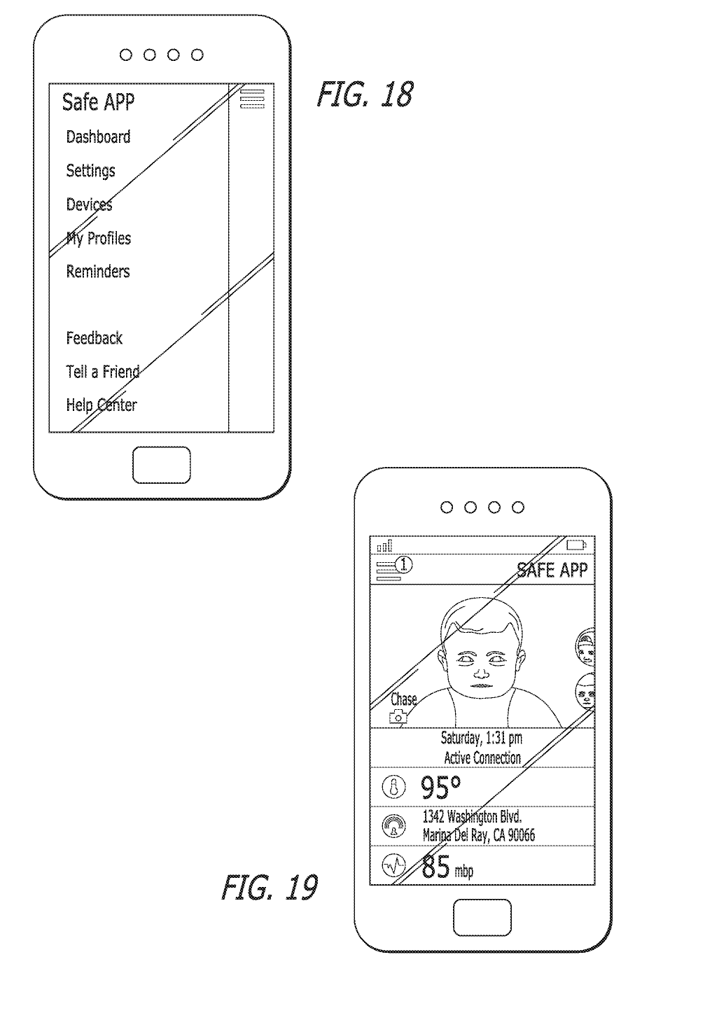

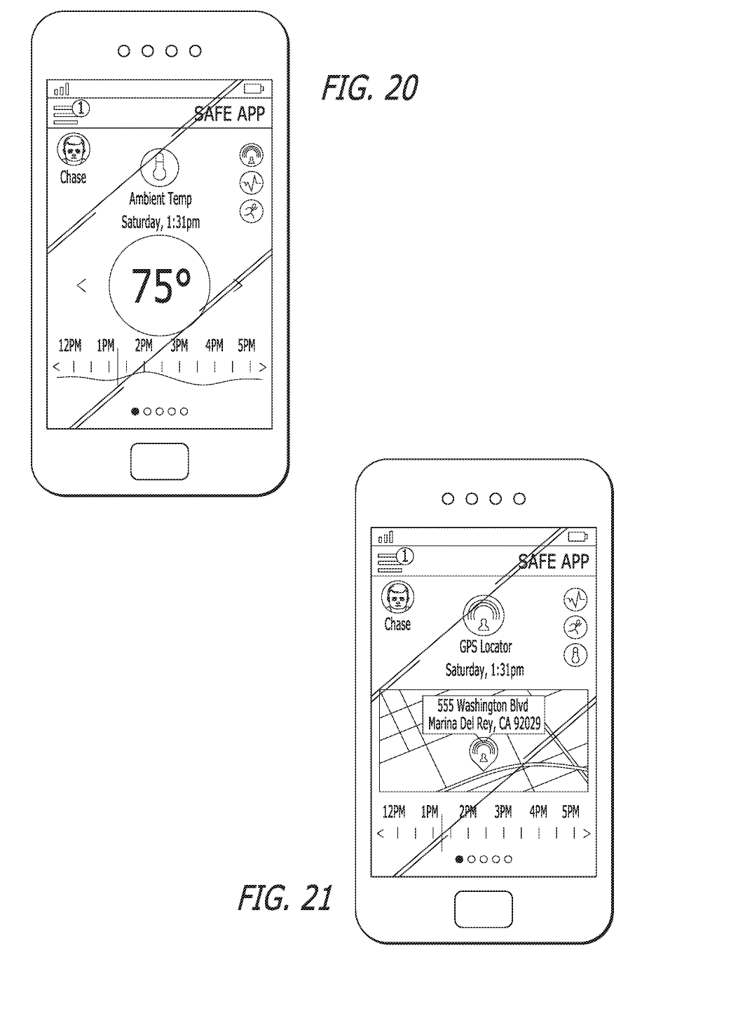

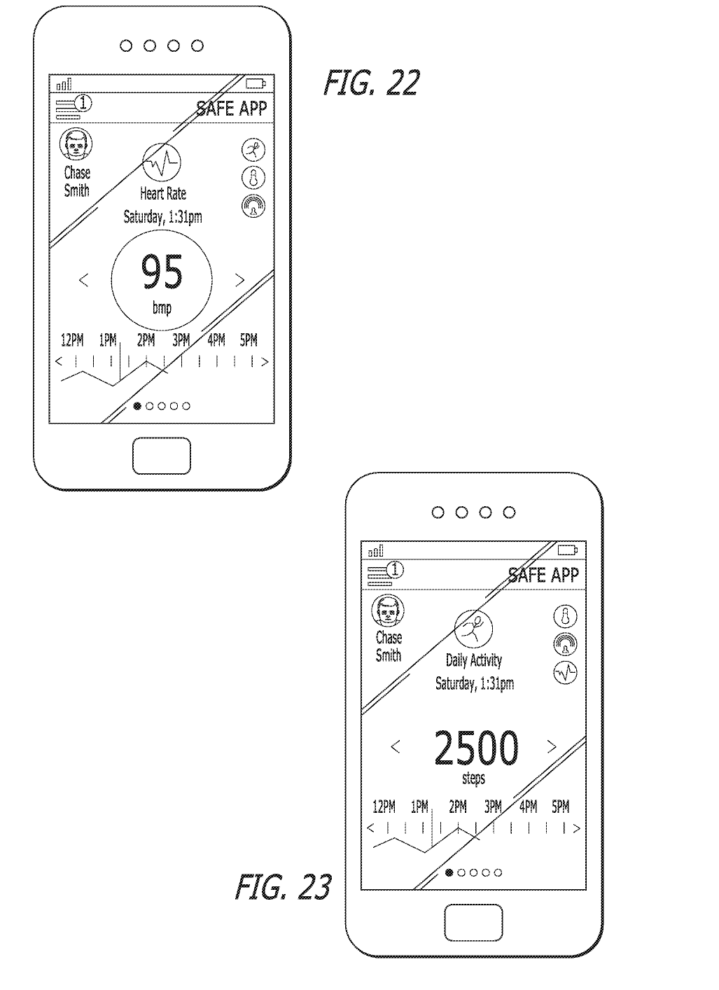

The buckle 5 is designed so that it is easiest to operate using two hands. The base housing 2 is made of flexible material and has an opening 13 that can be closed and locked shut with the buckle 5. On one side of the opening 13, an overlay clip 6 is attached to the base housing 2 via prongs 60(a), 60 (b) (FIG. 9(b)) that extend from the overlay clip 6 and engage prong holes 61(a), 61(b) (FIG. 10) in the housing cover 4. The overlay clip 6 has end mounts 62(a), 62(b) (FIG. 12) in which a rod 63 is mounted. The buckle 5 has a rod shaft 64 (FIG. 14) for receipt of the rod 63. The buckle is rotatably attached to the rod 63 by the rod shaft 64. A wire 74 (FIG. 14) is mounted in the buckle 5. The wire 74 has a generally U-shaped central section 65 (FIG. 14) and wire ends 66(a), 66(b) (FIG. 11) that engage wire holes 67(a), 67(b) (FIG. 14) in the end mounts 62(a), 62(b) (FIG. 12). The central section 65 is in contact with slider buttons 68(a), 68(b) (FIG. 14). When the slider buttons 68(a), 68(b) are squeezed toward one another, the wire ends 66(a), 66(b) are pulled out of engagement from the wire holes 67(a), 67(b). At this point, the buckle 5 is free to rotate about the rod 63. The buckle 5 has an inner surface 69 (FIG. 13) that faces the outer side 16 of the housing cover 4. Prongs 70(a), 70(b), 70(c), 70(d) (FIG. 12) extend from the inner surface 69 of the buckle 5 and engage prong holes 71(a), 71(b), 71(c), 71(d) (FIG. 7) in the housing cover 4. The buckle 5 has an open-side edge 72 (FIG. 14) opposite the rod shaft 64. To open the buckle 5, the slider buttons 68(a), 68(b) must be held in a squeezed-together state while at the same time the open-side edge 72 of the buckle 5 is pulled away from the housing cover 4 until the prongs 70(a), 70(b), 70(c), 70(d) are all free of the prong holes 71(a), 71(b), 71(c), 71(d).

The invention is described herein by way of example only and is not limited to the disclosed example(s). Similarly, the various figures, diagrams, dimensions, and photos are also provided as examples of the invention and to aid in understanding the invention. The invention is not limited to the examples shown in the referenced figures, diagrams, dimensions, and photos. The embodiments disclosed herein may be modified by those skilled in the art without departing from the scope of the invention.

Although the safety system of the present invention is particularly useful to monitor a child, it should be understood that it can be used to monitor a person of any age such as an infirm person or elderly person in need of constant care. The safety system can also be used to monitor a pet or other animal. Also, the safety system can be used to monitor a particular thing or location. For example, the wearable device can be modified to be attached to practically anything to keep track of the location of the thing or its surroundings. Things in this regard include without limitation suitcases, cars, boats, planes, computers, and other things. The wearable device can also be adapted to monitor the ambient conditions of any location where the wearable device is located or installed.

What is claimed is:

1. A wearable device comprising means to contemporaneously
(1) detect when a bluetooth signal between the wearable device and a smartphone is becoming weaker,
(2) detect when the ambient temperature is changing,
(3) detect whether activity of the person wearing the device has fallen below a specified threshold, and
(4) detect whether the GPS location is changing.

2. A wearable device as in claim 1, the wearable device further comprising means to make a telephone call.

3. A wearable device as in claim 1, the wearable device further comprising means to store a geographic boundary.

4. A wearable device as in claim 1, the wearable device further comprising means to store a specified ambient temperature.

5. A wearable device as in claim 1, the wearable device further comprising means to store a specified heart rate.

6. A wearable device as in claim 1, the wearable device further comprising a buckle.

7. A wearable device as in claim 1, the wearable device further comprising an anti-bacterial film.

8. A wearable device comprising means for
(1) detecting its global position,
(2) detecting the ambient temperature around the device,
(3) detecting the heart rate of a person wearing the wearable device,
(4) detecting the activity of the person wearing the wearable device, and
(5) making a telephone call for the purpose of sending information.

9. A wearable device as in claim 8, the wearable device further comprising means to store a geographic boundary.

10. A wearable device as in claim 8, the wearable device further comprising means to store a specified ambient temperature.

11. A wearable device as in claim 8, the wearable device further comprising means to store a specified heart rate.

12. A wearable device comprising means for
(1) sending a bluetooth signal,
(2) detecting the ambient temperature around the device,
(3) detecting the heart rate of a person wearing the wearable device,
(4) detecting the activity of the person wearing the wearable device, and
(5) making a telephone call for the purpose of sending information.

13. A wearable device as in claim 12, the wearable device further comprising means to store a specified ambient temperature.

14. A wearable device as in claim 12, the wearable device further comprising means to store a specified heart rate.
